(19) United States
(12) Patent Application Publication
(10) Pub. No.: US 2026/0069157 A1
(43) Pub. Date: Mar. 12, 2026

(54) METHOD FOR PROVIDING BIOLOGICAL INFORMATION AND WEARABLE ELECTRONIC DEVICE SUPPORTING SAME

(71) Applicant: Samsung Electronics Co., Ltd., Suwon-si (KR)

(72) Inventors: Younggeol KIM, Suwon-si (KR); Jindong KIM, Suwon-si (KR); Euiseok SHIN, Suwon-si (KR); Minhyun CHO, Suwon-si (KR)

(21) Appl. No.: 19/390,072

(22) Filed: Nov. 14, 2025

Related U.S. Application Data
(63) Continuation of application No. PCT/KR2024/006252, filed on May 9, 2024.

(30) Foreign Application Priority Data
May 23, 2023 (KR) 10-2023-0066159

(51) Int. Cl. A61B 5/026; A61B 5/00; A61B 5/02; A61B 5/021; A61B 5/024

(52) U.S. Cl. CPC A61B 5/0261; A61B 5/02007; A61B 5/021; A61B 5/02416; A61B 5/02438; A61B 5/681

(57) ABSTRACT

An electronic device may comprise a biometric sensor and at least one processor. At least one processor may be configured to obtain a PPG signal through the biometric sensor. At least one processor may be configured to obtain a blood flow rate of a user wearing a wearable electronic device on the basis of the PPG signal. At least one processor may be configured to obtain the blood pressure of the user. At least one processor may be configured to determine cardiovascular resistance indicating a degree to which blood flow is affected in a cardiovascular system of the user based on the obtained blood flow rate and the obtained blood pressure.

### CROSS-REFERENCE TO RELATED APPLICATIONS

This application is a continuation of International Application No. PCT/KR2024/006252 designating the United States, filed on May 9, 2024, in the Korean Intellectual Property Receiving Office and claiming priority to Korean Patent Application No. 10-2023-0066159, filed on May 23, 2023, in the Korean Intellectual Property Office, the disclosures of each of which are incorporated by reference herein in their entireties.

### BACKGROUND

### Field

The disclosure relates to a method for providing biological information and a wearable electronic device supporting the same.

### Description of Related Art

Electronic devices have been evolving into various forms for user convenience and are being miniaturized to be easily carried by the user.

Recently, with increasing interest in health, electronic devices are measuring biometric signals related to the human body and providing biological information based on the measured biometric signals. For example, electronic devices (e.g., wearable electronic devices) may obtain PPG signals through optical sensors (e.g., photoplethysmogram (PPG) sensors) for acquiring biometric signals and provide blood pressure based on the obtained PPG signals.

A user's blood pressure may be an indicator of the user's health. For example, a user's blood pressure may be associated with various diseases (e.g., heart diseases, strokes, heart failures, and retinal diseases).

However, even when a user's blood pressure is measured at the same value, the user's level of health may vary. For example, even when a user's blood pressure is measured at the same value of approximately 150 mmHg, the degree of vascular health of the user may vary depending on the diameter, thickness, and/or stiffness (or elasticity) of the blood vessels. Therefore, in addition to the user's blood pressure, a health indicator capable of more accurately reflecting the user's level of health may be needed.

### SUMMARY

Embodiments of the disclosure may provide to a method for providing biological information by obtaining cardiovascular resistance, indicating the degree to which blood flow is affected in the cardiovascular system, as a health indicator, based on a blood flow rate and blood pressure obtained based on a PPG signal, and a wearable electronic device supporting the method.

A wearable electronic device according to an example embodiment may include: a biometric sensor, at least one processor comprising processing circuitry, and memory storing instructions The instructions may cause, when executed by the at least one processor individually or collectively, the wearable electronic device to obtain a photoplethysmogram (PPG) signal through the biometric sensor; obtain a blood flow rate of a user wearing the wearable electronic device, based on the PPG signal; obtain blood pressure of the user; and determine, based on the obtained blood flow rate and blood pressure, cardiovascular resistance indicating a degree to which blood flow is affected in a cardiovascular system of the user.

According to an example embodiment, a method for providing biological information in a wearable electronic device may include: obtaining a photoplethysmogram (PPG) signal through a biometric sensor of the wearable electronic device; obtaining a blood flow rate of a user wearing the wearable electronic device, based on the PPG signal; obtaining blood pressure of the user; and determining, based on the obtained blood flow rate and blood pressure, cardiovascular resistance indicating a degree to which blood flow is affected in a cardiovascular system of the user.

According to an example embodiment, in a non-transitory computer-readable medium having computer-executable instructions recorded thereon, the computer-executable instructions, when executed by at least one processor, comprising processing circuitry, of a wearable electronic device, individually or collectively, cause the wearable electronic device to: obtain a photoplethysmogram (PPG) signal through a biometric sensor of the wearable electronic device; obtain a blood flow rate of a user wearing the wearable electronic device, based on the PPG signal; obtain blood pressure of the user; and determine, based on the obtained blood flow rate and blood pressure, cardiovascular resistance indicating a degree to which blood flow is affected in a cardiovascular system of the user.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments.

FIG. 2A is a front perspective view of an example wearable electronic device according to various embodiments.

FIG. 2B is a rear perspective view of the wearable electronic device shown in FIG. 2A according to various embodiments.

FIG. 2C is an exploded perspective view of the wearable electronic device shown in FIG. 2A according to various embodiments.

FIG. 3 is a block diagram illustrating an example configuration of a wearable electronic device according to various embodiments.

FIG. 4 is a diagram illustrating an example PPG sensor according to various embodiments.

FIG. 5 is a flowchart illustrating an example method for providing biological information according to various embodiments.

FIG. 6 is a flowchart illustrating an example method for obtaining a blood flow rate according to various embodiments.

FIG. 7 is a diagram illustrating an example method for obtaining a plurality of PPG signals according to various embodiments.

FIG. 8 is a diagram including graphs illustrating an example method for obtaining a time difference between a plurality of PPG signals according to various embodiments.

FIG. 9 is a diagram illustrating an example method for obtaining a plurality of PPG signals according to various embodiments.

FIG. 10 is a flowchart illustrating an example method for providing cardiovascular resistance according to various embodiments.

FIG. 11 is a graph illustrating an example method for providing cardiovascular resistance according to various embodiments.

### DETAILED DESCRIPTION

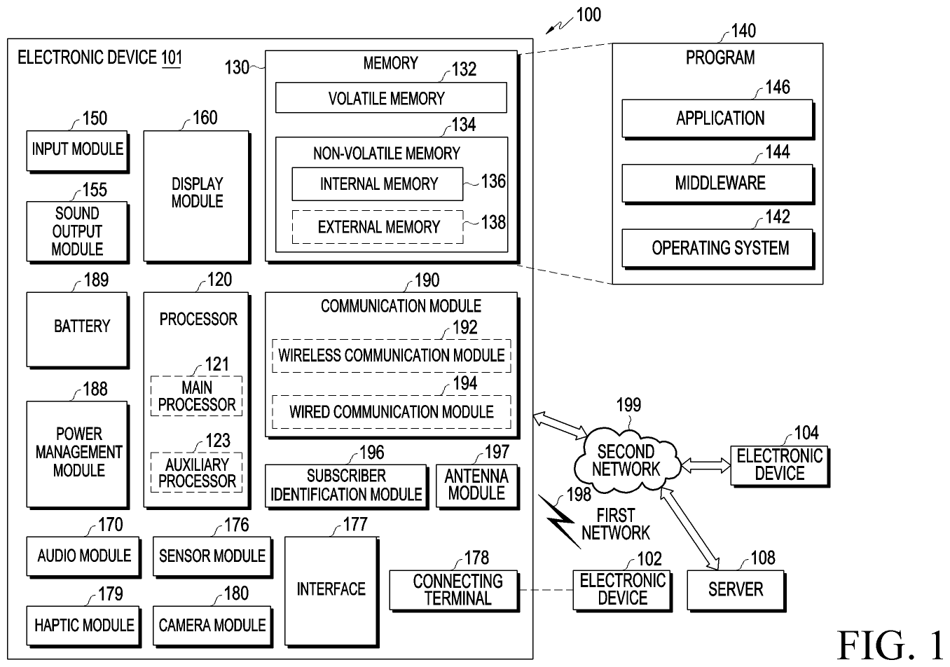

FIG. 1 is a block diagram illustrating an example electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In various embodiments, at least one of the components (e.g., the connecting terminal 178) may be omitted from the electronic device 101, or one or more other components may be added in the electronic device 101. In various embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121. Thus, the processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of Ims or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to an embodiment may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used in connection with an embodiment of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to an embodiment of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

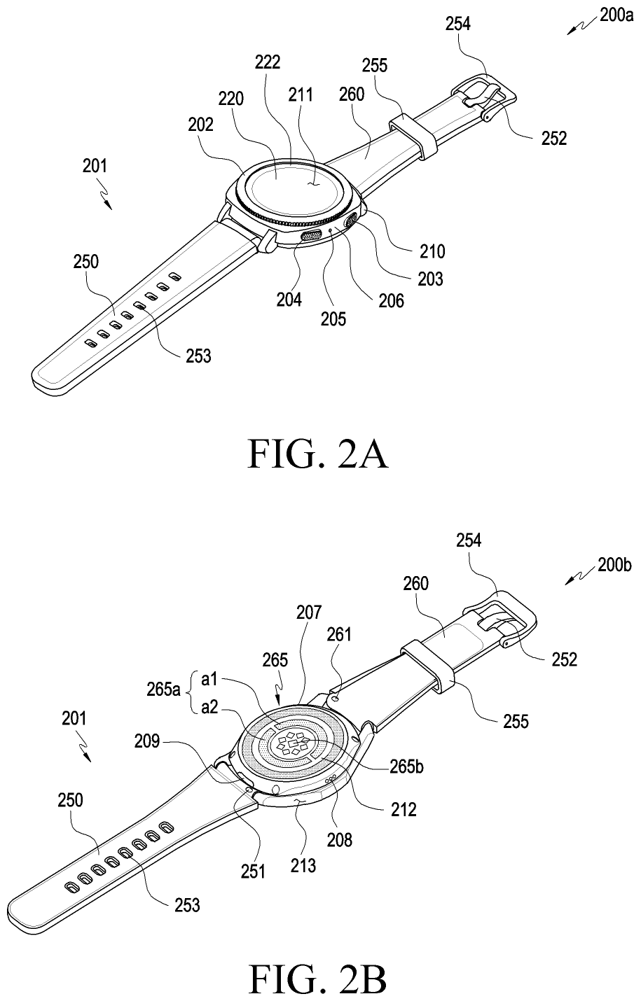

FIG. 2A is a front perspective view 200a of an example wearable electronic device 201 according to various embodiments.

FIG. 2B is a rear perspective view 200b of the wearable electronic device 201 shown in FIG. 2A according to various embodiments.

Referring to FIGS. 2A and 2B, a wearable electronic device 201 (e.g., the electronic device 101 in FIG. 1) according to an embodiment may include a housing 210 including a first surface (or front surface) 211, a second surface (or rear surface) 212, and a lateral surface 213 surrounding a space between the first surface 211 and the second surface 212, and wearing members 250 and 260 connected to at least a portion of the housing 210 and configured to detachably fasten the wearable electronic device 201 to a part of a user's body (e.g., a wrist or an ankle). In an embodiment (not shown), the housing 210 may also refer to a structure forming a portion of the first surface 211, the second surface 212, and the lateral surface 213 in FIGS. 2A and 2B. According to an embodiment, the first surface 211 may be formed by a front plate 222 (e.g., a glass plate or a polymer plate including various coating layers) that is at least partially transparent. The second surface 212 may be formed by a rear plate 207 that is substantially opaque. In various embodiments, in the case where the wearable electronic device 201 includes a sensor module 265 (e.g., the sensor module 176 in FIG. 1) disposed on the second surface 212 thereof, the rear plate 207 may include at least a partially transparent area.

The rear plate 207 may be formed of, for example, coated or tinted glass, ceramic, polymer, metal (e.g., aluminum, stainless steel (STS), or magnesium), or a combination of at least two of the above materials. The lateral surface 213 may be formed by a lateral bezel (or “lateral member”) 206 coupled to the front plate 222 and the rear plate 207 and including metal and/or polymer. In various embodiments, the rear plate 207 and the lateral bezel structure 206 may be integrally formed and may include the same material (e.g., a metal material such as aluminum). The wearing members 250 and 260 may be formed of various materials and configured in various shapes. They may be formed integrally or such that a plurality of unit links are movable relative to each other by a woven material, leather, rubber, urethane, metal, ceramic, or a combination of at least two of the above materials.

According to an embodiment, the wearable electronic device 201 may include at least one of a display 220 (see FIG. 2C) (e.g., the display module 160 in FIG. 1), audio modules 205 and 208 (e.g., the audio module 170 in FIG. 1), a sensor module 265 (e.g., the sensor module 176 in FIG. 1), key input devices 202, 203, and 204 (e.g., the input module 150 in FIG. 1), and a connector hole 209. In various embodiments, the wearable electronic device 201 may exclude at least one (e.g., the key input device 202, 203, or 204, the connector hole 209, or the sensor module 265) of the components, or may further include other components.

According to an embodiment, the wearable electronic device 201 may include a plurality of electrodes for measuring a biometric signal, and at least one of the plurality of electrodes may be disposed at a position of at least one of the key input devices 202, 203, and 204, the lateral bezel 206, the display 220, or the housing 210. Among the key input devices, a wheel key 202 may include a rotary bezel.

The display 220 may be visible, for example, through a substantial portion of the front plate 222. The display 220 may have various shapes corresponding to the shape of the front plate 222, such as a circular shape, an elliptical shape, or a polygonal shape. The display 220 may be connected to a touch detection circuit, a pressure sensor capable of measuring the intensity (pressure) of a touch, and/or a fingerprint sensor or disposed adjacent thereto.

According to an embodiment, the display 220 may include at least one transparent electrode for measuring a biometric signal among the plurality of electrodes for measuring a biometric signal.

The audio modules 205 and 208 may include a microphone hole 205 and a speaker hole 208. The microphone hole 205 may have a microphone disposed inside for obtaining external sound, and in various embodiments, a plurality of microphones may be disposed to detect the direction of sound. The speaker hole 208 may be used as an external speaker and a receiver for a call. In various embodiments, a speaker may be included without the speaker hole (e.g., a piezo speaker).

The sensor module 265 may generate electrical signals or data value corresponding to the internal operating state of the wearable electronic device 201 or the external environmental state. The sensor module 265, for example, a biometric sensor module 265 (e.g., HRM sensor) disposed on the second surface 212 of the housing 210 may include an ECG sensor 265a including at least two electrodes a1 and a2 for electrocardiogram measurement, and a PPG sensor 265b for heart rate measurement. The wearable electronic device 201 may further include a sensor module that is not shown, for example, at least one of a gesture sensor, a gyro sensor, a barometric pressure sensor, a magnetic sensor, an acceleration sensor, a grip sensor, a color sensor, an infrared (IR) sensor, a biometric sensor, a temperature sensor, a humidity sensor, or an illuminance sensor.

The key input devices 202, 203, and 204 may include a wheel key 202 disposed on the first surface 211 of the housing 210 and rotatable in at least one direction, and/or side key buttons 203 and 204 disposed on the lateral surface 213 of the housing 210. The wheel key 202 may have a shape corresponding to the shape of the front plate 222. In various embodiments, some of the key input devices 202, 203, and 204 may be implemented in other forms, such as soft keys on the display 220. The connector hole 209 may accommodate a connector (e.g., a USB connector) for transmitting and receiving power and/or data to and from an external electronic device, and may include another connector hole (not shown) to accommodate a connector for transmitting and receiving audio signals to and from the external electronic device. The wearable electronic device 201 may further include, for example, a connector cover (not shown) that covers at least a portion of the connector hole 209 and blocks the inflow of external foreign substances into the connector hole.

The wearing members 250 and 260 may be detachably fastened to at least a portion of the housing 210 using locking members 251 and 261. The locking members 251 and 261 may include fastening components, such as pogo pins, and may be replaced with protrusions or recesses formed in the wearing members 250 and 260 depending on the embodiment. For example, the wearing members 250 and 260 may be coupled to the housing 210 by engaging with the holes or protrusions formed in the housing 210. The wearing members 250 and 260 may include one or more of a fixing member 252, a fixing member-fastening hole 253, a band guide member 254, and a band fixing ring 255.

The fixing member 252 may be configured to secure the housing 210 and the wearing members 250 and 260 to a portion of the user's body (e.g., wrist or ankle). The fixing member-fastening hole 253, corresponding to the fixing member 252, may secure the housing 210 and the wearing members 250 and 260 to a portion of the user's body. The band guide member 254 may be configured to restrict the range of movement of the fixing member 252 when it is fastened to the fixing member-fastening hole 253, thereby ensuring that the wearing members 250 and 260 are securely fastened to a portion of the user's body. The band fixing ring 255 may restrict the range of movement of the wearing members 250 and 260 when the fixing member 252 and the fixing member-fastening hole 253 are fastened.

FIG. 2C is an exploded perspective view 200c of the wearable electronic device 201 shown in FIG. 2A according to various embodiments.

Referring to FIG. 2C, a wearable electronic device 201 (e.g., the electronic device 101 in FIG. 1) may include a lateral bezel structure 206, a wheel key 202, a front plate 222, a display 220, a first antenna 273, a support member 274 (e.g., a bracket), a battery 277, a first printed circuit board 281 (e.g., a printed circuit board (PCB), a printed board assembly (PBA), a flexible PCB (FPCB), or a rigid-flexible PCB (RFPCB)), a second printed circuit board 282, a sealing member 279, a second antenna 278, a rear housing 207, a rear cover 283, a signal detecting unit 284 (e.g., the electrode 265a of the ECG or BIA sensor, and the PPG sensor 265b in FIG. 2B), and fastening members 250 and 260. At least one of the components of the wearable electronic device 201 may be identical or similar to at least one of the components of the wearable electronic device 201 shown in FIG. 2A or FIG. 2B, and redundant descriptions thereof will be omitted below.

In an embodiment, the support member 274 may be disposed inside the wearable electronic device 201 and connected to the lateral bezel structure 206, or may be formed integrally with the lateral bezel structure 206. The support member 274 may be formed of, for example, a metal material and/or a non-metal material (e.g., polymer). The support member 274 may have a display 220 coupled to one surface thereof and a first printed circuit board 281 coupled to the opposite surface. The printed circuit board 281 may have a processor (e.g., the processor 120 in FIG. 1), memory (e.g., the memory 130 in FIG. 1), and/or an interface (e.g., the interface 177 in FIG. 1), which are mounted thereon. The processor may include, for example, one or more of a central processing unit, an application processor, a graphics processing unit (GPU), an application signal processor, or a communication processor.

In an embodiment, the memory may include volatile memory (e.g., the volatile memory 132 in FIG. 1) or non-volatile memory (e.g., the non-volatile memory 134 in FIG. 1). The interface may include, for example, a high-definition multimedia interface (HDMI), a universal serial bus (USB) interface, an SD card interface, and/or an audio interface.

In an embodiment, the interface may electrically or physically connect the wearable electronic device 201, for example, to an external electronic device, and include a USB connector, an SD card/MMC connector, or an audio connector.

In an embodiment, the battery 277 may be a device for supplying power to at least one component of the wearable electronic device 201, and may include, for example, a non-rechargeable primary battery, a rechargeable secondary battery, or a fuel cell. At least a portion of the battery 277 may be disposed substantially on the same plane as, for example, the printed circuit board 281. The battery 277 may be integrally disposed within the wearable electronic device 201, or may be detachably disposed in the wearable electronic device 201.

In an embodiment, the first antenna 273 may be disposed between the display 220 and the support member 274. The first antenna 273 may include, for example, a near-field communication (NFC) antenna, a wireless charging antenna, and/or a magnetic secure transmission (MST) antenna. For example, the first antenna 273 may perform short-range communication with an external device, wirelessly transmit and receive power required for charging, and transmit magnetic-based signals including short-range communication signals or payment data. In an embodiment, the antenna structure may be formed by a part or combination of the lateral bezel structure 206 and/or the support member 274.

In an embodiment, the second antenna 278 may be disposed between the circuit board 281 and the rear plate 207. The second antenna 278 may include, for example, a near-field communication (NFC) antenna, a wireless charging antenna, and/or a magnetic secure transmission (MST) antenna. For example, the second antenna 278 may perform short-range communication with an external device, wirelessly transmit and receive power required for charging, and transmit magnetic-based signals including short-range communication signals or payment data. In an embodiment, the antenna structure may be formed by a part or combination of the lateral bezel structure 206 and/or the rear plate 207.

In an embodiment, the sealing member 279 may be disposed between the lateral bezel structure 206 and the rear plate 207. The sealing member 279 may be configured to block moisture and foreign matter entering the space enclosed by the lateral bezel structure 206 and the rear plate 207 from the outside. In addition, the sealing member 279 may shield electromagnetic signals. For example, the sealing member 279 may perform a shielding function to shield electromagnetic interference (EMI) or other various electrical signals.

In an embodiment, the rear housing 207 and the rear cover 283 may support various components included in the wearable electronic device 201. The rear housing 207 and the rear cover 283 may be included, for example, in the rear plate 207 described above in FIG. 2B.

In an embodiment, the rear cover 283 may be formed of a transparent material such that light is able to pass through at least a portion. For example, a sensor (not shown) disposed on the second printed circuit board 282 may include a light emitter capable of generating light and a light receiver capable of receiving light. The light emitter may emit light to the outside through a portion of the rear cover 283 formed of a transparent material, and the light receiver may receive external light through a portion of the rear cover 283 formed of a transparent material. For example, the sensor including the light emitter and the light receiver may be a sensor that measures blood flow using photoplethysmography (PPG), thereby measuring information related to a user's heart rate.

In an embodiment, the signal detecting unit 284 may include an electrode (e.g., the ECG or BIA sensor electrode 265a in FIG. 2B) that comes into contact with the user's body. For example, the signal detecting unit 284 may be formed at least partially on a portion of the rear cover 283 that may come into contact with the user's body.

In an embodiment, the second printed circuit board 282 may include at least one of the various components of the electronic device described above in FIG. 1. In an embodiment, the second printed circuit board 282 may be electrically connected to the first printed circuit board 281 described above. In an embodiment, the internal electronic components of the electronic device may be distributed and arranged on the first printed circuit board 281 and the second printed circuit board 282. In an embodiment, the second printed circuit board 282 may be connected to the signal detecting unit 284, and may receive and process a signal detected by the signal detecting unit 284. In various embodiments, a sensing processing circuit or microcontroller (MCU) distinct from the processor that controls the overall operation of the wearable electronic device 201 may be disposed on the second printed circuit board 282 and may independently/primarily process a signal detected by a sensor (e.g., a PPG sensor) disposed on the second printed circuit board and/or the signal detecting unit 284.

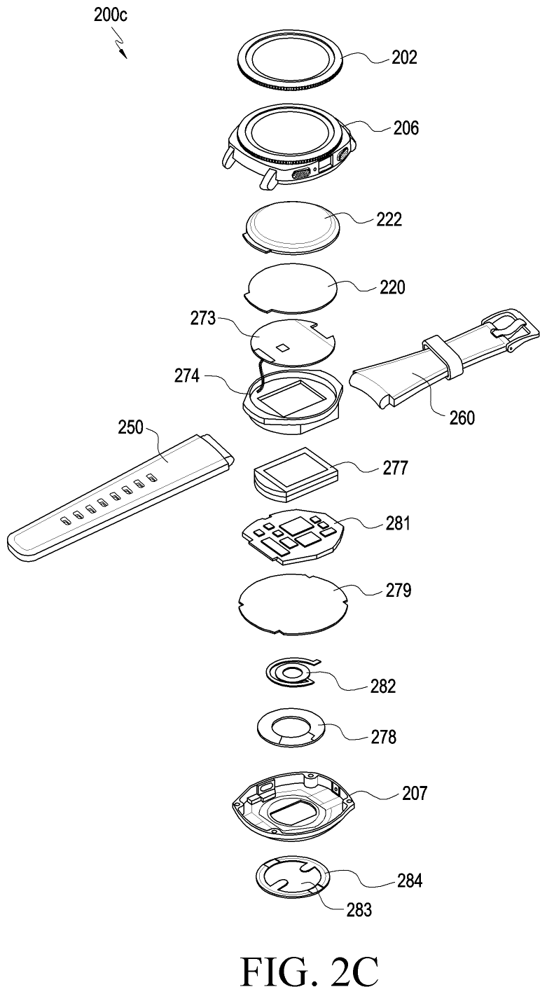

FIG. 3 is a block diagram illustrating an example configuration of a wearable electronic device 301 according to various embodiments.

Referring to FIG. 3, in an embodiment, a wearable electronic device 301 may be the electronic device 101 described with reference to FIG. 1 and/or the wearable electronic device 201 described with reference to FIGS. 2A, 2B, and 2C.

In an embodiment, the wearable electronic device 301 may be an electronic device including at least one of the components of the electronic device 101 in FIG. 1 and/or at least one of the components of the wearable electronic device 201 in FIGS. 2A, 2B, and 2C.

In an embodiment, the wearable electronic device 301 may include a communication module (e.g., including communication circuitry) 310, a display module (e.g., including a display) 320, a biometric sensor 330, memory 340, and/or a processor (e.g., including processing circuitry) 350.

In an embodiment, the communication module 310 may be the communication module 190 in FIG. 1. The communication module 310 may include various communication circuitry and support communication between the wearable electronic device 301 and an external electronic device.

In an embodiment, the communication module 310 may receive information about the user's blood pressure from an external electronic device. For example, the external electronic device may be a blood pressure measurement device (e.g., a cuff-type blood pressure measurement device, a mechanical blood pressure measurement device, or an electronic blood pressure measurement device) capable of measuring the user's blood pressure. The communication module 310 may receive information about the user's blood pressure measured by the blood pressure measurement device from the blood pressure measurement device.

In an embodiment, the display module 320 may be the display module 160 in FIG. 1 or the display 220 in FIG. 2A (and FIG. 2C).

In an embodiment, the display module 320 may include a display and display information related to biological information. The information related to biological information displayed by the display module 320 will be described in detail later.

In an embodiment, the biometric sensor 330 may be included in the sensor module 176 in FIG. 1 or the sensor module 265 in FIG. 2B.

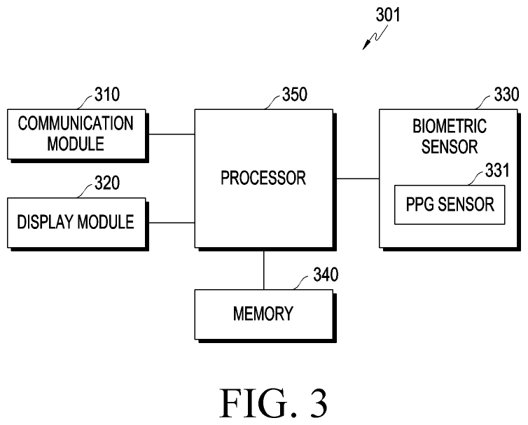

In an embodiment, the biometric sensor 330 may include a photoplethysmogram (PPG) sensor 331 (e.g., the PPG sensor 265b in FIG. 2B). Hereinafter, the PPG sensor 331 will be described in greater detail below with reference to FIG. 4.

FIG. 4 is a diagram illustrating an example PPG sensor 331 according to various embodiments.

Referring to FIG. 4, in an embodiment, the PPG sensor 331 may include at least one light emitter, a plurality of light receivers, and a signal processor, each of which may include various circuitry.

In an embodiment, as indicated by reference numeral 401 in FIG. 4, the PPG sensor 331 may include a light emitter 410 and two light receivers 421 and 422 (and a signal processor (not shown)).

In an embodiment, two light receivers 421 and 422 may be disposed on the wearable electronic device 301 so as to be substantially parallel to the direction in which the user's blood flows (or the direction of the blood vessels) (e.g., the direction indicated by the arrow 450) when the wearable electronic device 301 is worn on the user (e.g., the user's wrist).

In an embodiment, as indicated by reference numeral 402 in FIG. 4, the PPG sensor 331 may include a light emitter 430 and eight light receivers 441, 442, 443, 444, 445, 446, 447, and 448 (and a signal processor (not shown)).

Although reference numerals 401 and 402 in FIG. 4 illustrate that the PPG sensor 331 includes two or eight light receivers, the disclosure is not limited thereto. In addition, although reference numerals 401 and 402 in FIG. 4 illustrate that the PPG sensor 331 includes one light emitter 410, the disclosure is not limited thereto. For example, the PPG sensor 331 may include two or more light emitters.

In an embodiment, the light emitter (e.g., the light emitter 410 or the light emitter 430) may output light to the user's skin. The light emitter may sequentially or simultaneously output at least one of infrared rays, red, green, and/or blue light. The light emitter may include at least one of a spectrometer, a vertical cavity surface emitting laser (VCSEL), a light-emitting diode (LED), a white LED, and a white laser.

In an embodiment, the light receiver (e.g., the light receivers 421 and 422 or the light receivers 441, 442, 443, 444, 445, 446, 447, and 448) may receive light (or light signals) transmitted from the outside. For example, the light receiver may receive at least a portion of light (or light signals), among the light output from the light emitter, that is reflected by a user's body tissue (e.g., skin, skin tissue, fat layer, veins, arteries, and/or capillaries). The light receiver may output a signal corresponding to the received light. For example, the light receiver may include at least one of an avalanche photodiode (APD), a single photon avalanche diode (SPAD), a photodiode, a photomultiplier tube (PMT), a charge coupled device (CCD), a CMOS array, or a spectrometer.

In an embodiment, a signal processor included in the PPG sensor 331 may process biometric signals detected from the plurality of light receivers. In an embodiment, the signal detecting unit (e.g., an analog front end) may include an amplifier for amplifying a PPG signal and an analog-to-digital converter (ADC) for converting an analog PPG signal into a digital PPG signal. However, the components included in the signal processor are not limited to the aforementioned amplifier and ADC.

Referring to FIG. 3, in an embodiment, the biometric sensor 330 may include additional sensors, in addition to the PPG sensor 331. For example, the biometric sensor 330 may further include a sensor that may measure a biometric signal (e.g., an electrocardiogramaignal, a galvanic skin response (GSR) signal, an electroencephalogram (EEG) signal, or a bio-electrical impedance analysis (BIA) signal) using one or more electrodes.

In FIG. 3, although the wearable electronic device 301 is illustrated as including the biometric sensor 330, the wearable electronic device 301 may further include at least one sensor included in the sensor module 176 in FIG. 1 and/or at least one sensor included in the sensor module 265 in FIG. 2B.

In an embodiment, the memory 340 may be the memory 130 in FIG. 1.

In an embodiment, the memory 340 may store information for performing an operation of providing biological information. The information, stored in the memory 340, for the operation of providing biological information will be described in detail later.

In an embodiment, the processor 350 may be the processor 120 in FIG. 1, and the detailed description of processor 120 applies equally to the processor 350.

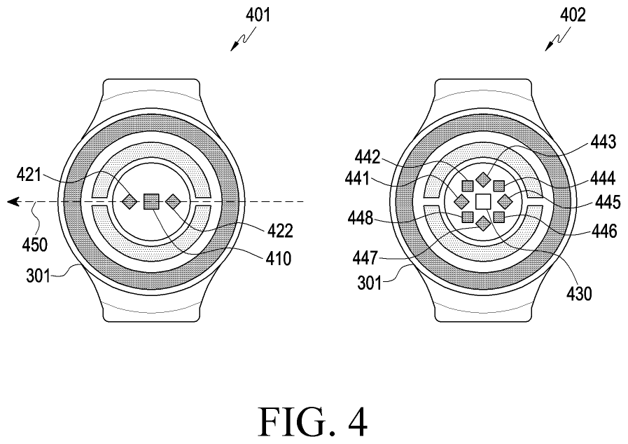

In an embodiment, the processor 350 may control the overall operation of providing biological information. In an embodiment, the processor 350 may include one or more processors for providing biological information. The operation performed by the processor 350 to provide biological information will be described in greater detail below with reference to FIG. 5 and subsequent drawings.

Although the wearable electronic device 301 is illustrated as including the communication module 310, the display module 320, the biometric sensor 330, the memory 340, and the processor 350 in FIG. 3, the disclosure is not limited thereto. For example, the wearable electronic device 301 may further include at least one component included in the electronic device 101 in FIG. 1 and/or the wearable electronic device 201 in FIG. 2.

FIG. 5 is a flowchart 500 illustrating an example method for providing biological information according to various embodiments.

Referring to FIG. 5, in operation 501, in an embodiment, the processor 350 may obtain a PPG signal through the biometric sensor 330.

In operation 503, in an embodiment, the processor 350 may obtain a blood flow rate of a user, based on the obtained PPG signal.

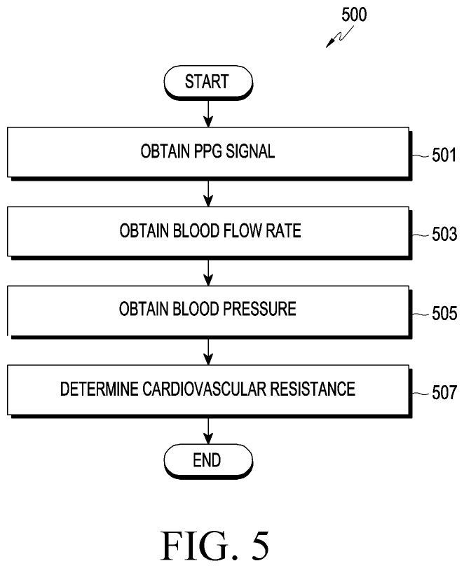

Hereinafter, an operation of obtaining a PPG signal through the biometric sensor 330 and an operation of obtaining a user's blood flow rate, based on the obtained PPG signal, will be described in greater detail below with reference to FIGS. 6 to 9.

FIG. 6 is a flowchart 600 illustrating an example method for obtaining a blood flow rate according to various embodiments.

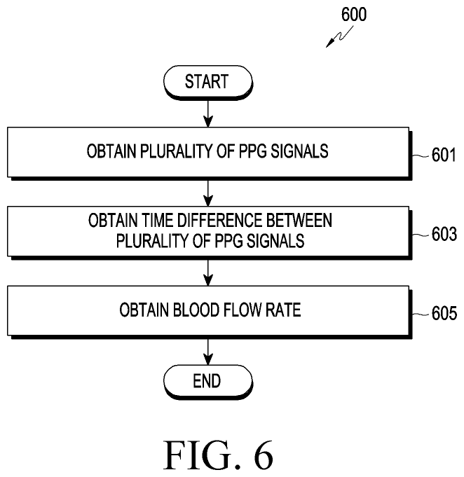

FIG. 7 is a diagram illustrating an example method for obtaining a plurality of PPG signals according to various embodiments.

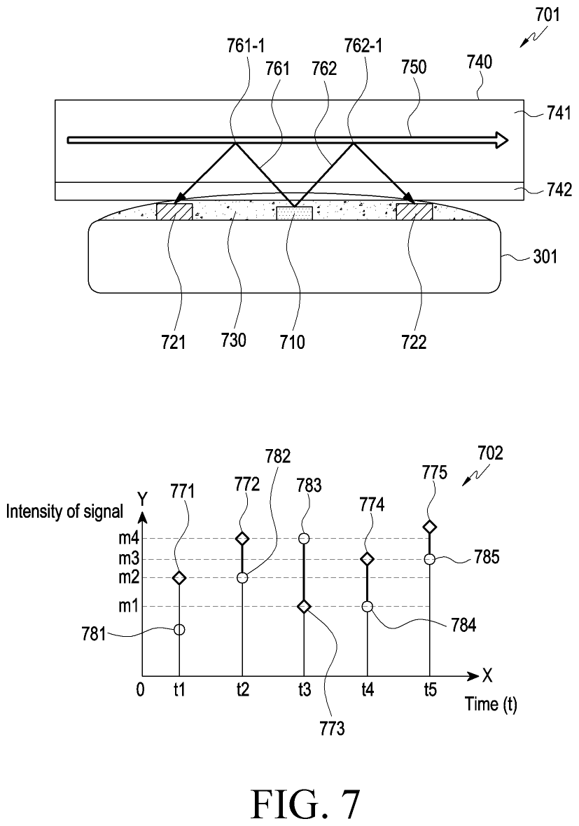

FIG. 8 is a diagram including graphs illustrating an example method for obtaining a time difference between a plurality of PPG signals according to various embodiments.

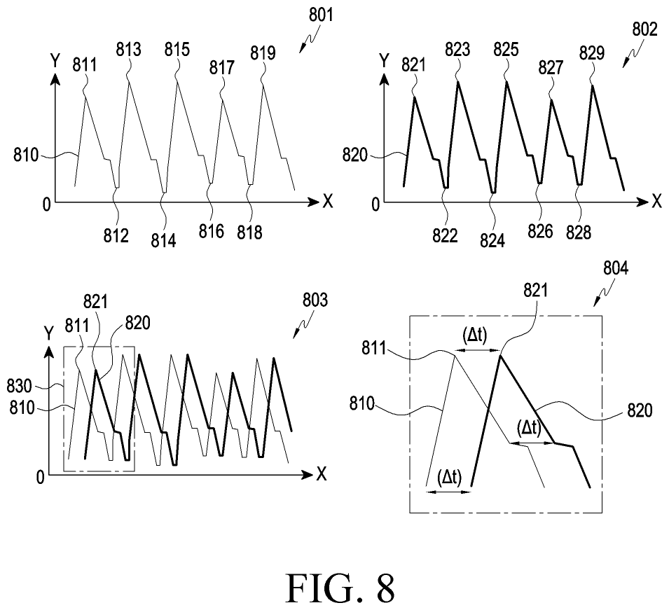

FIG. 9 is a diagram 900 illustrating an example method for obtaining a plurality of PPG signals according to various embodiments.

Referring to FIGS. 6 to 9, in operation 601, in an embodiment, the processor 350 may obtain a plurality of PPG signals through the PPG sensor 331.

In an embodiment, the PPG sensor 331 may include at least one light emitter and a plurality of light receivers. For example, as indicated by reference numeral 701 in FIG. 7, the PPG sensor 331 may include one light emitter (e.g., a light emitter 710), and a plurality of light receivers (e.g., a first light receiver 721 and a second light receiver 722).

In an embodiment, the first light receiver 721 and the second light receiver 722 may correspond to the light receiver 422 and the light receiver 421, respectively, indicated by reference numeral 401 in FIG. 4. However, the disclosure is not limited thereto, and for example, the first light receiver 721 and the second light receiver 722 may correspond to two light receivers among the plurality of light receivers 441, 442, 443, 444, 445, 446, 447, and 448 indicated by reference numeral 402 in FIG. 4. As indicated by reference numeral 701, the PPG sensor 331 is illustrated as including two light receivers (e.g., a first light receiver 721 and a second light receiver 722), but the disclosure is not limited thereto. For example, the PPG sensor 331 may include three or more light receivers.

In an embodiment, as indicated by reference numeral 701, the processor 350 may control the light emitter 710 to emit light 761 and 762. For example, the processor 350 may control the light emitter 710 to periodically emit red light or infrared rays.

In an embodiment, the light 761 and light 762 emitted from the light emitter 710 may pass through the epidermal layer 742 of the skin 740 of the user wearing the wearable electronic device 301, pass through the blood vessels of the dermal layer 741, and then be reflected. The reflected light 761 and light 762 may be detected (e.g., received) by the first light receiver 721 and the second light receiver 722, respectively. The first light receiver 721 and the second light receiver 722 may generate a plurality of PPG signals by detecting the light 761 and light 762, respectively. Hereinafter, for convenience of explanation, the PPG signal generated by the first light receiver 721 will be referred to as a “first PPG signal” and the PPG signal generated by the second light receiver 722 will be referred to as a “second PPG signal.” In the diagram indicated by reference numeral 701, reference numeral 730 may represent a glass member of the wearable electronic device 301.

In an embodiment, as indicated by reference numeral 701, an arrow 750 may represent blood in a blood vessel, and the direction indicated by the arrow 750 may represent the direction of the blood flow in the blood vessel. In the diagram indicated by reference numeral 701, the blood may flow through the blood vessel from the left side to the right side of the user's skin 740, as indicated by the arrow 750. In an embodiment, two light receivers 721 and 722 may be disposed on the wearable electronic device 301 so as to be substantially parallel to the direction in which the user's blood flows (or the direction of the blood vessel) (e.g., the direction indicated by the arrow 750) when the wearable electronic device 301 is worn on the user (e.g., the user's wrist).

In an embodiment, after a predetermined time has elapsed from the point in time at which a first PPG signal is generated, a second PPG signal having the same waveform as the first PPG signal may be generated. For example, as indicated by the arrow 750 in the diagram indicated by reference numeral 701, in the case where the user's blood flows from the left side to the right side of the user's skin 740, light 761 may be transmitted through the blood passing through a first position 761-1 of the blood vessel, then reflected, and detected by the first light receiver 721 at a first point in time. The blood that passed through the first position 761-1 of the blood vessel at the first point in time may pass through a second position 762-1 of the blood vessel at a second point in time after a predetermined period of time has elapsed from the first point in time. The second light receiver 722 may detect light 762 that is transmitted through the blood passing through the second position 762-1 of the blood vessel and then reflected at the second point in time. The first light receiver 721 and the second light receiver 722 may detect light that is transmitted through the same portion of the blood flowing through the blood vessel and then reflected at different points in time, such as the first point in time and the second point in time, respectively, thereby generating the first PPG signal and the second PPG signal of the same waveform with a time difference.

In an embodiment, the values of the first PPG signal generated by the light 761 detected at the first point in time may be respectively the same as the values of the second PPG signal generated by the light 762 detected at the second point in time after a predetermined period of time has elapsed from the first point in time.

In an embodiment, the second PPG signal generated by the light 762 detected at the second point in time after a predetermined period of time has elapsed from the first point in time may be a signal that has the same waveform as that of the first PPG signal generated by the light 761 detected at the first point in time, and is measured with a delay relative to the first PPG signal by an amount of time obtained by subtracting the first point in time from the second point in time on the time axis.

In an embodiment, in the diagram indicated by reference numeral 702 in FIG. 7, the X-axis may represent time, and the Y-axis may represent the intensity of the PPG signal. As indicated by reference numeral 702, as time (t) elapses, values 771, 772, 773, 774, and 775 of the first PPG signal may be measured at each of times t1, t2, t3, t4, and t5 through the first light receiver 721. As time (t) elapses, values 781, 782, 783, 784, and 785 of the second PPG signal may be measured at each of times t1, t2, t3, t4, and t5 through the second light receiver 722.

In an embodiment, as described above, the values of the first PPG signal generated at the first point in time may be identical to the values of the second PPG signal generated at the second point in time after a predetermined period of time has elapsed from the first point in time. For example, the value 782 (e.g., m2) of the second PPG signal measured at time t2 may be equal to the value 771 of the first PPG signal measured at time t1. The value 783 (e.g., m4) of the second PPG signal measured at time t3 may be equal to the value 772 of the first PPG signal measured at time t2. The value 784 (e.g., m1) of the second PPG signal measured at time t4 may be equal to the value 773 of the first PPG signal measured at time t3. The value 785 (e.g., m3) of the second PPG signal measured at time t5 may be equal to the value 774 of the first PPG signal measured at time t4.

In operation 603, in an embodiment, the processor 350 may obtain a time difference between the plurality of PPG signals. For example, the processor 350 may calculate a time difference between a time at which values of the first PPG signal are measured and a time at which values of the second PPG signal that are equal to the values of the first PPG signal are measured (hereinafter referred to as a “time difference between the plurality of PPG signals”) (e.g., a time difference between the first point in time and the second point in time described in operation 601).

In an embodiment, the processor 350 may obtain a time difference between the plurality of PPG signals by comparing the plurality of PPG signals. For example, the processor 350 may obtain a time difference between the plurality of PPG signals by comparing peak values and/or valley values of the plurality of PPG signals (e.g., waveforms of the plurality of PPG signals) with each other. For example, the processor 350 may obtain a time difference between the plurality of PPG signals, based on the similarity (or consistency) between the plurality of PPG signals.

In an embodiment, reference numeral 801 in FIG. 8 may represent a graph indicating a waveform 810 of the first PPG signal, and reference numeral 802 in FIG. 8 may represent a graph indicating a waveform of the second PPG signal 820. In the graphs indicated by reference numerals 801, 802, and 803, the X-axis may represent time, and the Y-axis may represent the intensity of the PPG signal (or the value of the PPG signal).

In an embodiment, in the graph indicated by reference numeral 801, points 811, 813, 815, 817, and 819 may represent peaks of a waveform 810 of the first PPG signal, respectively, and points 812, 814, 816, and 818 may represent valleys of the waveform 810 of the first PPG signal, respectively.

In an embodiment, in the graph indicated by reference numeral 802, points 821, 823, 825, 827, and 829 may represent peaks of a waveform 820 of the second PPG signal, respectively, and points 822, 824, 826, and 828 may represent valleys of the waveform 820 of the second PPG signal, respectively.

In an embodiment, the points 811, 813, 815, 817, 819 representing peaks of the waveform 810 of the first PPG signal may correspond to the points 821, 823, 825, 827, and 829 representing peaks of the waveform 820 of the second PPG signal, respectively. The points 812, 814, 816, and 818 representing valleys of the waveform 810 of the first PPG signal may correspond to the points 822, 824, 826, and 828 representing valleys of the waveform 820 of the second PPG signal, respectively.

In an embodiment, reference numeral 803 may be a graph that represents both the waveform 810 of the first PPG signal and the waveform 820 of the second PPG signal. For example, reference numeral 803 may be a graph that shows the waveform 810 of the first PPG signal and the waveform 820 of the second PPG signal overlaid over time.

In an embodiment, reference numeral 804 may be a graph that shows a part 830 of the graph indicated by reference numeral 803 enlarged.

In an embodiment, the processor 350 may obtain a time difference between the plurality of PPG signals by comparing the peak values and/or valley values of the waveforms of the plurality of PPG signals. For example, in the graphs indicated by reference numerals 803 and 804, the processor 350 may identify the time at which the peak 811 of the waveform 810 of the first PPG signal is measured and the time at which the peak 821 of the waveform 820 of the second PPG signal corresponding to the peak 811 of the waveform 810 of the first PPG signal is measured. The processor 350 may obtain (e.g., calculate) the time difference Δt between the peak 811 of the waveform 810 of the first PPG signal and the peak 821 of the waveform 820 of the second PPG signal by subtracting the time at which the peak 811 of the waveform 810 of the first PPG signal is measured from the time at which the peak 821 of the waveform 820 of the second PPG signal is measured. For example, in the graphs indicated by reference numerals 803 and 804, the processor 350 may identify the time at which the valley of the waveform 810 of the first PPG signal is measured and the time at which the valley of the waveform 820 of the second PPG signal corresponding to the valley of the waveform 810 of the first PPG signal is measured. The processor 350 may obtain (e.g., calculate) the time difference Δt between the valley of the waveform 810 of the first PPG signal and the valley of the waveform 820 of the second PPG signal by subtracting the time at which the valley of the waveform 810 of the first PPG signal is measured from the time at which the valley of the waveform 820 of the second PPG signal is measured.

In an embodiment, the time difference Δt between the peak 811 of the waveform 810 of the first PPG signal and the peak 821 of the waveform 820 of the second PPG signal may be substantially the same as the time difference Δt between the valley of the waveform 810 of the first PPG signal and the valley of the waveform 820 of the second PPG signal. The processor 350 may determine the time difference Δt between the peak 811 of the waveform 810 of the first PPG signal and the peak 821 of the waveform 820 of the second PPG signal, or the time difference Δt between the valley of the waveform 810 of the first PPG signal and the valley of the waveform 820 of the second PPG signal, as the time difference between the plurality of PPG signals.

In an embodiment, the processor 350 may obtain a time difference between the plurality of PPG signals, based on similarity (or consistency) between the plurality of PPG signals. For example, the processor 350 may obtain a time difference between the first PPG signal and the second PPG signal by comparing the waveform 810 of the first PPG signal and the waveform 820 of the second PPG signal as a whole. For example, the processor 350 may shift the waveform 810 of the first PPG signal along the time axis (e.g., the X-axis) so that the waveform 810 of the first PPG signal substantially overlaps the waveform 820 of the second PPG signal. When the waveform 810 of the first PPG signal substantially overlaps the waveform 820 of the second PPG signal according to the shift along the time axis, the processor 350 may obtain, as a time difference between the first PPG signal and the second PPG signal, the period of time (e.g., Δt) by which the waveform 810 of the first PPG signal is moved so that the waveform 810 of the first PPG signal substantially overlaps the waveform 820 of the second PPG signal.

In operation 605, in an embodiment, the processor 350 may obtain (e.g., calculate) a blood flow rate, based on the time difference between the plurality of PPG signals and the distances between the plurality of light receivers of the PPG sensor 331.

In an embodiment, the distances between the plurality of light receivers of the PPG sensor 331 may be stored in the memory 340. The processor 350 may obtain the distances between the plurality of light receivers of the PPG sensor 331 from the memory 340. For example, the processor 350 may obtain the distance between the first light receiver 721 and the second light receiver 722 from the memory 340.

In an embodiment, the processor 350 may obtain the blood flow rate by dividing the distance between the plurality of light receivers of the PPG sensor 331 by the time difference between the plurality of PPG signals. For example, the processor 350 may calculate the blood flow rate, as the speed at which blood flows in a blood vessel, by dividing the distance between the first light receiver 721 and the second light receiver 722 by the time difference (e.g., Δt) between the second PPG signal and the first PPG signal. However, the disclosure is not limited thereto. For example, as illustrated in FIG. 7, the distance between the first light receiver 721 and the second light receiver 722 may be different from the distance between the first position 761-1 of the blood vessel from which light 761 is reflected and the second position 762-1 of the blood vessel from which light 762 is reflected. The processor 350 may calculate a ratio (e.g., approximately 0.5) of the first position 761-1 of the blood vessel from which light 761 is reflected and the second position 762-1 of the blood vessel from which light 762 is reflected to the distance between the first light receiver 721 and the second light receiver 722. The processor 350 may calculate the blood flow rate by dividing the product of the distance between the first light receiver 721 and the second light receiver 722 and the calculated ratio (e.g., a value obtained by multiplying the distance between the first light receiver 721 and the second light receiver 722 by approximately 0.5) by the time difference (e.g., Δt) between the second PPG signal and the first PPG signal.

Although the operation of obtaining the blood flow rate using the PPG sensor 331 including a single light emitter (e.g., the light emitter 710) and a plurality of light receivers (e.g., the first light receiver 721 and the second light receiver 722) has been described with reference to reference numeral 701 in FIG. 7 above, the disclosure is not limited thereto.

In an embodiment, as illustrated in FIG. 9, the PPG sensor 331 may include a plurality of light emitters (e.g., a first light emitter 911 and a second light emitter 912) and a plurality of light receivers (e.g., a first light receiver 921 and a second light receiver 922).

In an embodiment, in FIG. 9, the processor 350 may control the first light emitter 911 and the second light emitter 912 to emit light 961 and 962, respectively. For example, the processor 350 may control the first light emitter 911 and the second light emitter 912 to periodically emit red light or infrared rays.

In an embodiment, in FIG. 9, an arrow 950 may represent blood in a blood vessel, and the direction indicated by the arrow 950 may represent the direction of the blood flow in the blood vessel. As illustrated in FIG. 9, the blood may flow through the blood vessel from the left side to the right side of the user's skin 940, as indicated by the arrow 950.

In an embodiment, light 961 and light 962 respectively emitted from the first light emitter 911 and the second light emitter 912 may pass through the epidermal layer 942 of the skin 940 of the user wearing the wearable electronic device 301, pass through the blood vessels of the dermal layer 941, and then be reflected. The reflected light 961 and light 962 may be detected (e.g., received) by the first light receiver 921 and the second light receiver 922, respectively. The first light receiver 921 and the second light receiver 922 may generate a plurality of PPG signals by detecting the light 961 and light 962, respectively. In an embodiment, in FIG. 9, reference numeral 930 may represent a glass member of the wearable electronic device 301.

In an embodiment, the processor 350 may obtain a time difference between the plurality of PPG signals generated from the plurality of light receivers (e.g., the first light receiver 921 and the second light receiver 922).

In an embodiment, the processor 350 may obtain a blood flow rate by dividing the distance between the plurality of light receivers (e.g., the first light receiver 921 and the second light receiver 922) of the PPG sensor 331 by the time difference between the plurality of PPG signals. For example, the processor 350 may calculate the blood flow rate, as the speed at which blood flows in a blood vessel, by dividing the distance between the first light receiver 921 and the second light receiver 922 by the time difference (e.g., Δt) between the second PPG signal and the first PPG signal.

In an embodiment, the processor 350 may obtain a more precise time difference between the plurality of PPG signals by increasing the operating frequency of the PPG sensor 331, for example, the operating frequency of at least one light emitter (e.g., the frequency at which at least one light emitter emits light) and the operating frequencies (or sampling frequencies) of the plurality of light receivers.

Referring to FIG. 5, in an embodiment, in operation 505, the processor 350 may obtain the blood pressure of a user (e.g., a user wearing the wearable electronic device 301).

In an embodiment, the processor 350 may receive, from an external electronic device, information about the user's blood pressure, which is obtained (e.g., measured) by the external electronic device, via the communication module 310. For example, the processor 350 may receive information about the user's blood pressure, obtained (e.g., measured) by a blood pressure measurement device (e.g., a cuff-type blood pressure measurement device, a mechanical blood pressure measurement device, or an electronic blood pressure measurement device), from the blood pressure measurement device via the communication module 310.

In an embodiment, the processor 350 may receive, from the external electronic device, information about the user's blood pressure, obtained (e.g., measured) by the external electronic device, via the communication module 310 while performing operations 501 and 503. For example, while the processor 350 performs operations 501 and 503 to obtain a blood flow rate, the user may measure blood pressure in real time through a cuff-type blood pressure measurement device. When blood pressure measurement is completed through the cuff-type blood pressure measurement device while performing the operations to obtain a blood flow rate, the processor 350 may receive information about the measured blood pressure from the cuff-type blood pressure measurement device through the communication module 310. However, the disclosure is not limited thereto. For example, the processor 350 may perform an operation of receiving, from the external electronic device, information about the user's blood pressure obtained by the external electronic device before or after performing operations 501 and 503 to obtain a blood flow rate.

In an embodiment, the processor 350 may obtain the user's blood pressure through a biometric sensor 330 (e.g., the PPG sensor 331). For example, the processor 350 may identify a previously obtained PPG signal and user's blood pressure (e.g., a PPG signal and a user's blood pressure obtained at the same previous point in time). The processor 350 may compare the PPG signal currently obtained through the PPG sensor 331 with the identified PPG signal (e.g., the previously obtained PPG signal) and compensate for the identified blood pressure (e.g., the previously obtained blood pressure), thereby obtaining the user's blood pressure.

In an embodiment, the user's blood pressure may include the magnitude of the systolic blood pressure of the user. However, the disclosure is not limited thereto, and the user's blood pressure may include the magnitude of the diastolic blood pressure and/or the magnitude of the systolic blood pressure of the user.

In operation 507, in an embodiment, the processor 350 may determine, based on the obtained blood flow rate and blood pressure, cardiovascular resistance indicating the degree to which blood flow is affected in the user's cardiovascular system.

In an embodiment, cardiovascular resistance may be a health indicator that may indicate the degree to which blood flow is affected in the user's cardiovascular system.

In an embodiment, cardiovascular resistance may be related to the stiffness (or elasticity) of blood vessels and the extent to which thrombi and/or fatty calcifications are deposited in blood vessels (e.g., blood vessel walls). For example, when the blood vessel stiffness increases, the cardiovascular resistance may also increase. For example, the blood vessel diameter may decrease when thrombi and/or fatty calcifications are deposited in the blood vessels, and this decrease in blood vessel diameter may increase the cardiovascular resistance.

In an embodiment, the cardiovascular resistance may be related to the blood flow rate and blood pressure. For example, when the same blood pressure is measured, an increase in cardiovascular resistance (e.g., a decrease in blood vessel diameter or an increase in blood vessel stiffness) may increase the blood flow rate. In addition, an increase in blood pressure may increase the blood flow rate. Accordingly, the cardiovascular resistance may be expressed using the blood flow rate and blood pressure, as shown in Equation 1 below.

Cardiovascular resistance=k*(blood flow)/(blood pressure) [Equation 1]

In an embodiment, in Equation 1, k may be a coefficient. For example, k may be a coefficient that reflects factors affecting the cardiovascular resistance, in addition to the blood flow rate and blood pressure. In an embodiment, the cardiovascular resistance may be calculated by dividing the blood flow rate by the blood pressure.

In an embodiment, a lower cardiovascular resistance may indicate better health for the user, while a higher cardiovascular resistance may indicate worse health for the user.

Although not illustrated in FIG. 5, in an embodiment, the processor 350 may provide information related to the blood flow rate, the blood pressure, and/or the cardiovascular resistance. For example, the processor 350 may display the blood flow rate, the blood pressure, and/or the cardiovascular resistance through the display module 320. For example, the processor 350 may display information about cardiovascular system diseases related to the blood flow rate, the blood pressure, and/or the cardiovascular resistance through the display module 320.

Although not illustrated in FIG. 5, in an embodiment, the processor 350 may transmit the information related to the blood flow rate, the blood pressure, and/or the cardiovascular resistance to an external electronic device (e.g., a smartphone wirelessly connected to the wearable electronic device 301) via the communication module 310 so that the external electronic device may display the information related to the blood flow rate, the blood pressure, and/or the cardiovascular resistance.

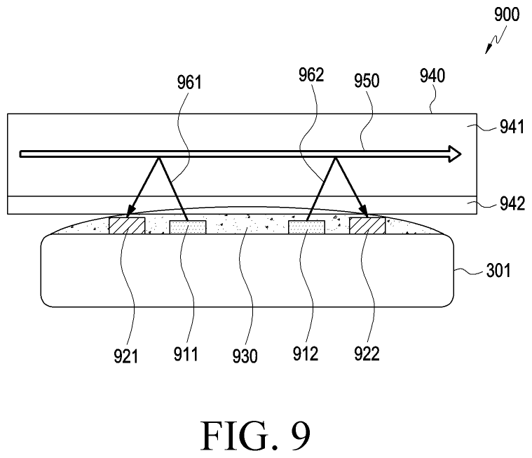

FIG. 10 is a flowchart 1000 illustrating an example method for providing cardiovascular resistance according to various embodiments.

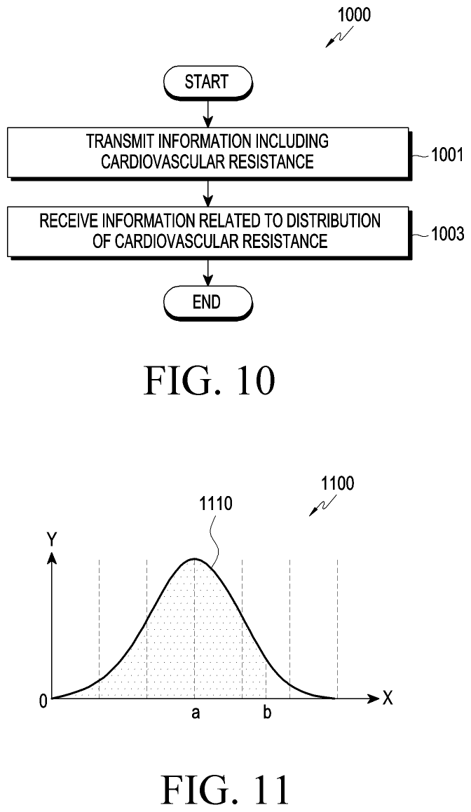

FIG. 11 is a graph 1100 illustrating an example method for providing cardiovascular resistance according to various embodiments.

Referring to FIGS. 10 and 11, in operation 1001, in an embodiment, the processor 350 may control or cause the wearable electronic device 301 to transmit information including cardiovascular resistance to a server (e.g., the server 108 in FIG. 1) through the communication module 310. For example, the processor 350 may transmit information including cardiovascular resistance and user information (e.g., the user's age and/or gender) to the server through the communication module 310.

In an embodiment, the server may receive, from a plurality of electronic devices including the wearable electronic device 301, a plurality of cardiovascular resistances of a plurality of users respectively corresponding to the plurality of electronic devices.

In an embodiment, the server may obtain a distribution of cardiovascular resistances according to age groups of the plurality of users, based on the plurality of cardiovascular resistances of the plurality of users. For example, the server may calculate the mean and standard deviation of cardiovascular resistances by age group of the plurality of users. Based on the calculated mean and standard deviation of cardiovascular resistances, the server may calculate a normal distribution of cardiovascular resistances by age group of the plurality of users.

In an embodiment, the server may determine, based on the calculated normal distribution and the cardiovascular resistance of each of the plurality of users within the same age group, a percentage (or rank, or score) corresponding to the user's cardiovascular resistance among the cardiovascular resistances of the plurality of users within the same age group. For example, when the user of the wearable electronic device 301 is in the 40s, the server may determine, based on the calculated normal distribution and the user's cardiovascular resistance, that the user's cardiovascular resistance falls within the top 30% of the cardiovascular resistances of the plurality of users in their 40s.

In an embodiment, the server may transmit information regarding the percentage corresponding to the user's cardiovascular resistance among the calculated normal distribution and/or the cardiovascular resistances of the plurality of users to the wearable electronic device 301.

In operation 1003, in an embodiment, the processor 350 may control or cause the wearable electronic device 301 to receive information related to the distribution of cardiovascular resistance from the server through the communication module 310. For example, the processor 350 may receive, from the server, the information regarding the percentage corresponding to the user's cardiovascular resistance among the calculated normal distribution and/or the cardiovascular resistances of the plurality of users through the communication module 310.

In an embodiment, the processor 350 may cause the wearable electronic device 301 to display information related to the user's cardiovascular resistance through a display, based on the received information. For example, the processor 350 may display a graph 1110 representing a normal distribution related to cardiovascular resistance in the user's age group, a value (a) representing the average cardiovascular resistance in the user's age group, a value (b) representing the user's cardiovascular resistance, and a percentage corresponding to the user's cardiovascular resistance (e.g., top 30%), as shown in FIG. 11.

Although FIGS. 10 and 11 illustrate that the server obtains a distribution of cardiovascular resistance by age group for the plurality of users, based on the plurality of cardiovascular resistances of the plurality of users, the disclosure is not limited thereto. For example, the wearable electronic device 301 may perform at least some of the operations of the server described above.

A wearable electronic device 301 according to an example embodiment may include a biometric sensor 330, at least one processor 350, and memory 340 storing instructions. The instructions may cause, when executed by the at least one processor 350 individually or collectively, the wearable electronic device 301 to obtain a PPG signal through the biometric sensor 330. The instructions may cause, when executed by the at least one processor 350 individually or collectively, the wearable electronic device 301 to obtain a blood flow rate of a user wearing the wearable electronic device 301, based on the PPG signal. The instructions may cause, when executed by the at least one processor 350 individually or collectively, the wearable electronic device 301 to obtain blood pressure of the user. The instructions may cause, when executed by the at least one processor 350 individually or collectively, the wearable electronic device 301 to determine, based on the obtained blood flow rate and blood pressure, cardiovascular resistance indicating a degree to which blood flow is affected in a cardiovascular system of the user.

In an example embodiment, the biometric sensor 330 may include a PPG sensor 331 including at least one light emitter and a plurality of light receivers. The instructions may cause, when executed by the at least one processor 350 individually or collectively, the wearable electronic device 301 to obtain a plurality of PPG signals through the respective light receivers.

In an example embodiment, the plurality of light receivers may include a first light receiver and a second light receiver. The first light receiver and the second light receiver may be disposed on the wearable electronic device 301 so as to be substantially parallel to a direction in which a blood of the user flows, when the wearable electronic device 301 is worn on the user.

In an example embodiment, the instructions may cause, when executed by the at least one processor 350 individually or collectively, the wearable electronic device 301 to obtain a time difference between the plurality of PPG signals by comparing the plurality of PPG signals, obtain a distance between the plurality of light receivers, and obtain the blood flow rate, based on the distance and the time difference.

In an example embodiment, based on the plurality of PPG signals including a first PPG signal generated through a first light receiver and a second PPG signal generated through a second light receiver, the waveform of the second PPG signal may be same as the waveform of the first PPG signal, and the second PPG signal may be generated after the time difference from a point in time at which the first PPG signal is generated.

In an example embodiment, the wearable electronic device 301 may further include a communication module 310. The instructions may cause, when executed by the at least one processor 350 individually or collectively, the wearable electronic device 301 to receive a blood pressure of the user measured by an external electronic device from the external electronic device through the communication module 310.

In an example embodiment, the cardiovascular resistance may be related to stiffness of blood vessels of the user, a degree to which thrombi are deposited in the blood vessels, and/or a degree to which fatty calcifications are deposited in the blood vessels.

In an example embodiment, the instructions may cause, when executed by the at least one processor 350 individually or collectively, the wearable electronic device 301 to determine the cardiovascular resistance by dividing the blood flow rate by the blood pressure.

In an example embodiment, the wearable electronic device 301 may further include a communication module 310. The instructions may cause, when executed by the at least one processor 350 individually or collectively, the wearable electronic device 301 to transmit information regarding the cardiovascular resistance to a server through the communication module 310, and receive information related to a distribution of the cardiovascular resistance from the server through the communication module 310.

In an example embodiment, the information related to the distribution of the cardiovascular resistance may include a percentage corresponding to the cardiovascular resistance of the user among the cardiovascular resistances of a plurality of users of a same age as the user.

In an example embodiment, a method for providing biological information in a wearable electronic device 301 may include obtaining a PPG signal through a biometric sensor 330 of the wearable electronic device 301. The method may include obtaining a blood flow rate of a user wearing the wearable electronic device 301, based on the PPG signal. The method may include obtaining blood pressure of the user. The method may include determining, based on the obtained blood flow rate and blood pressure, cardiovascular resistance indicating the degree to which blood flow is affected in a cardiovascular system of the user.

In an example embodiment, the biometric sensor 330 may include a PPG sensor 331 including at least one light emitter and a plurality of light receivers. The obtaining of the PPG signal may include obtaining a plurality of PPG signals through the plurality of light receivers, respectively.

In an example embodiment, the plurality of light receivers may include a first light receiver and a second light receiver. The first light receiver and the second light receiver may be disposed on the wearable electronic device 301 so as to be substantially parallel to a direction in which a blood of the user flows, when the wearable electronic device 301 is worn on the user.

In an example embodiment, the obtaining of the blood flow rate may include obtaining a time difference between the plurality of PPG signals by comparing the plurality of PPG signals, obtaining a distance between the plurality of light receivers, and obtaining the blood flow rate, based on the distance and the time difference.

In an example embodiment, based on the plurality of PPG signals including a first PPG signal generated through a first light receiver and a second PPG signal generated through a second light receiver, a waveform of the second PPG signal is same as a waveform of the first PPG signal, and the second PPG signal may be generated after the time difference from a point in time at which the first PPG signal is generated.

In an example embodiment, the obtaining of the blood pressure of the user may include receiving a blood pressure of the user measured by an external electronic device from the external electronic device through the communication module 310.

In an example embodiment, the cardiovascular resistance may be related to stiffness of the user's blood vessels, a degree to which thrombi are deposited in the blood vessels, and/or a degree to which fatty calcifications are deposited in the blood vessels.

In an example embodiment, the determining of the cardiovascular resistance may include determining the cardiovascular resistance by dividing the blood flow rate by the blood pressure.

In an example embodiment, the method may further include transmit information regarding the cardiovascular resistance to a server through a communication module 310 of the wearable electronic device 301, and receiving information related to a distribution of the cardiovascular resistance from the server through the communication module 310.

In an example embodiment, the information related to the distribution of the cardiovascular resistance may include a percentage corresponding to the cardiovascular resistance of the user among the cardiovascular resistances of a plurality of users of the same age as the user.

Additionally, the data structure used in the various embodiments of the disclosure described above may be recorded on a computer-readable recording medium through various means. The computer-readable recording medium includes storage media such as magnetic storage media (e.g., ROM, floppy disks, hard disks, etc.) and optical reading media (e.g., CD-ROMs, DVDs, etc.).

While the disclosure has been illustrated and described with reference to various example embodiments, it will be understood that the various example embodiments are intended to be illustrative, not limiting. It will be further understood by those skilled in the art that various modifications, alternatives and/or variations of the various example embodiments may be made without departing from the true technical spirit and full technical scope of the disclosure, including the appended claims and their equivalents. It will also be understood that any of the embodiment(s) described herein may be used in conjunction with any other embodiment(s) described herein.

What is claimed is:

1. A wearable electronic device comprising:
a biometric sensor;
at least one processor comprising processing circuitry; and
memory storing instructions that, when executed by the at least one processor individually or collectively, cause the wearable electronic device to:
obtain a photoplethysmogram (PPG) signal through the biometric sensor,
obtain a blood flow rate of a user wearing the wearable electronic device, based on the PPG signal,
obtain blood pressure of the user, and
based on the obtained blood flow rate and blood pressure, determine cardiovascular resistance indicating a degree to which blood flow is affected in a cardiovascular system of the user.

2. The wearable electronic device of claim 1,
wherein the biometric sensor comprises a PPG sensor comprising at least one light emitter and a plurality of light receivers, and
wherein the instructions, when executed by the at least one processor individually or collectively, cause the wearable electronic device to obtain a plurality of PPG signals through the plurality of light receivers, respectively.

3. The wearable electronic device of claim 2,
wherein the plurality of light receivers comprise a first light receiver and a second light receiver, and
wherein the first light receiver and the second light receiver are disposed on the wearable electronic device to be substantially parallel to a direction in which a blood of the user flows, based on the wearable electronic device being worn on the user.

4. The wearable electronic device of claim 2,
wherein the instructions, when executed by the at least one processor individually or collectively, cause the wearable electronic device to:
obtain a time difference between the plurality of PPG signals by comparing the plurality of PPG signals,
obtain a distance between the plurality of light receivers, and
obtain the blood flow rate, based on the distance and the time difference.

5. The wearable electronic device of claim 4,
wherein, based on the plurality of PPG signals including a first PPG signal generated through a first light receiver and a second PPG signal generated through a second light receiver, a waveform of the second PPG signal is same as a waveform of the first PPG signal, and the second PPG signal is generated after the time difference from a point in time at which the first PPG signal is generated.

6. The wearable electronic device of claim 1, further comprising a communication module comprising communication circuitry,
wherein the instructions, when executed by the at least one processor individually or collectively, cause the wearable electronic device to receive the blood pressure of the user measured by an external electronic device from the external electronic device through the communication module.

7. The wearable electronic device of claim 1,
wherein the cardiovascular resistance is related to stiffness of blood vessels of the user, a degree to which thrombi are deposited in the blood vessels, and/or a degree to which fatty calcifications are deposited in the blood vessels.

8. The wearable electronic device of claim 1,
wherein the instructions, when executed by the at least one processor individually or collectively, cause the wearable electronic device to determine the cardiovascular resistance by dividing the blood flow rate by the blood pressure.

9. The wearable electronic device of claim 1,
further comprising a communication module comprising communication circuitry,
wherein the instructions, when executed by the at least one processor individually or collectively, further cause the wearable electronic device:
transmit information regarding the cardiovascular resistance to a server through the communication module, and
receive information related to a distribution of the cardiovascular resistance from the server through the communication module.

10. The wearable electronic device of claim 9,
wherein the information related to the distribution of the cardiovascular resistance comprises a percentage corresponding to the cardiovascular resistance of the user among the cardiovascular resistances of a plurality of users of a same age as the user.

11. A method for providing biological information in a wearable electronic device, the method comprising:
obtaining a photoplethysmogram (PPG) signal through a biometric sensor of the wearable electronic device;
obtaining a blood flow rate of a user wearing the wearable electronic device, based on the PPG signal;
obtaining blood pressure of the user; and
based on the obtained blood flow rate and blood pressure, determining cardiovascular resistance indicating a degree to which blood flow is affected in a cardiovascular system of the user.

12. The method of claim 11,
wherein the biometric sensor comprises a photoplethysmogram (PPG) sensor comprising at least one light emitter and a plurality of light receivers, and
wherein the obtaining of the PPG signal comprises
obtaining a plurality of PPG signals through the plurality of light receivers, respectively.

13. The method of claim 12,
wherein the plurality of light receivers comprise a first light receiver and a second light receiver, and
wherein the first light receiver and the second light receiver are disposed on the wearable electronic device to be substantially parallel to a direction in which a blood of the user flows, when the wearable electronic device is worn on the user.

14. The method of claim 12,
wherein the obtaining of the blood flow rate comprises:
obtaining a time difference between the plurality of PPG signals by comparing the plurality of PPG signals;
obtaining a distance between the plurality of light receivers; and
obtaining the blood flow rate, based on the distance and the time difference.

15. The method of claim 14,
wherein, based on the plurality of PPG signals including a first PPG signal generated through a first light receiver and a second PPG signal generated through a second light receiver, a waveform of the second PPG signal is same as a waveform of the first PPG signal, and the second PPG signal is generated after the time difference from a point in time at which the first PPG signal is generated.

16. The method of claim 11, wherein the obtaining of the blood pressure of the user comprises:
receiving the blood pressure of the user measured by an external electronic device from the external electronic device through a communication module of the wearable electronic device.

17. The method of claim 11, wherein the cardiovascular resistance is related to stiffness of blood vessels of the user, a degree to which thrombi are deposited in the blood vessels, and/or a degree to which fatty calcifications are deposited in the blood vessels.

18. The method of claim 11, wherein the determining of the cardiovascular resistance comprises:
determining the cardiovascular resistance by dividing the blood flow rate by the blood pressure.

19. The method of claim 11, further comprising:
transmitting information regarding the cardiovascular resistance to a server through a communication module, and
receiving information related to a distribution of the cardiovascular resistance from the server through the communication module of the wearable electronic device.

20. The method of claim 19, wherein the information related to the distribution of the cardiovascular resistance comprises a percentage corresponding to the cardiovascular resistance of the user among the cardiovascular resistances of a plurality of users of a same age as the user.
